(19) United States
(12) Patent Application Publication
(10) Pub. No.: US 2012/0226159 A1
(43) Pub. Date: Sep. 6, 2012

(54) ULTRASONIC SCANNING SYSTEM AND ULTRASOUND IMAGE ENHANCEMENT METHOD

(76) Inventors: Anthony Sinclair, Toronto (CA); Luke Wesley, Toronto (CA); Maciej Jastrzebski, Oakville (CA); Tom Dusatko, Vancouver (CA); Joel Fortin, Montreal (CA); Farhang Honavar, Tehran (IR)

(21) Appl. No.: 13/319,454

(22) PCT Filed: May 8, 2009
(86) PCT No.: PCT/CA2009/000644

§ 371 Date: May 21, 2012

(51) Int. Cl. A61B 8/14

(52) U.S. Cl. 600/443

(57) ABSTRACT

An ultrasonic measurement system comprises a processor configured for enhancing a received ultrasonic signal, where the received ultrasonic signal is a frequency domain signal. The enhancing involves deconvolving the received ultrasonic signal to yield a filtered signal, determining autoregressive extrapolation parameters based on frequency amplitude fluctuations of the filtered signal within a frequency range over which a corresponding reference signal has a high signal-to-noise ratio, and carrying out an autoregressive spectral extrapolation of the filtered signal using the autoregressive extrapolation parameters to yield an enhanced ultrasonic signal.

### FIELD OF THE INVENTION

The present invention relates generally to ultrasonic scanning and particularly, to an ultrasonic scanning system and method of enhancing an ultrasound image.

### BACKGROUND OF THE INVENTION

Non-destructive imaging techniques are employed in numerous fields to survey interior sections of materials a quick and facile way. In the field of oil and gas pipelines, for example, ultrasonic scanning has proven to be an economical way to survey large sections of pipelines and is often used to identify potential defects in pipeline walls, which can originate during fabrication and construction of the pipelines. Pipeline defects often occur at the girth welds joining adjacent sections of the pipeline. Consequently, during pipeline construction each girth weld is ultrasonically scanned immediately after fabrication to ensure that the girth weld has properly penetrated the entire pipeline wall thickness, is free of cracks, and has the necessary strength and structural integrity to withstand the internal pressures in the pipeline. The process of pipeline construction only proceeds after each fresh girth weld has passed a safety inspection and is determined to be defect free. Welding equipment is typically not moved until the safety inspection has been completed and as a result, this inspection process creates a bottleneck in pipeline construction.

Time of flight diffraction (“TOFD”) is a commonly used method of determining the size and position of defects in girth welds from ultrasound scans. The TOFD method is illustrated in FIG. 1 and utilizes a transmitter probe 32 for emitting ultrasonic signals and a receiver probe 34 for receiving ultrasonic signals arranged in a “pitch-catch” configuration. The orientation of the two probes 32 and 34 enables a large cross sectional area of a specimen 36, in this case adjacent pipeline sections joined by a girth weld 38, to be scanned. In this example, girth weld 38 includes a defect 40 in the form of a crack that has formed perpendicularly to the direction of maximum stress, which is the typical orientation of girth weld cracks as is known in the art. The time of flight of each ultrasonic signal echo can be determined from analysis of the ultrasonic signal received by the receiver probe 34. As shown in FIG. 2, the first component or echo of the ultrasonic signal to arrive at the receiver probe 34 is the strong “lateral” wave corresponding to the shortest path between the two probes 32 and 34. Similarly, the last ultrasonic signal component to arrive at the receiver probe 34 is the ultrasonic signal that reflects off of the backwall of the pipeline section (i.e. the wall furthest from the probes 32 and 34), corresponding to the longest distance the ultrasonic signal travels between the two probes. Moving the pair of probes 32 and 34 circumferentially around the pipeline section allows an ultrasonic image of the circumferential volume of the pipeline section and girth weld 38 to be mapped, from which the position and size of any defects within the girth weld 38 can be determined, as shown in FIG. 3.

The TOFD method, however, is known to have limitations. Firstly, the detection of small cracks using TOFD is inherently difficult, as the multiple ultrasound signal echoes from the extremities of a short crack tend to overlap and become indistinguishable from one another. Specifically, ultrasonic signal echoes originating from the top and bottom tips of a defect cannot be resolved if the defect is less than approximately 3 millimeters in length. In such cases, the ultrasonic signal echoes from each tip will overlap to yield a combined ultrasonic signal that will suggest the presence of a defect, but for which a precise sizing of the defect is not possible. Secondly, TOFD produces significant “dead zones”, particularly near the frontwall and backwall of the pipeline section, where crack tip ultrasonic signal echoes can be obscured by the strong lateral wave and by backwall ultrasound signal echoes. While other diffraction methods, such as the back-diffraction technique, in which an ultrasonic scan is performed using a phased-array probe positioned in a pulse-echo configuration in relation to the specimen, have been shown to be capable of measuring crack sizes of 2 mm, this method is best suited in cases where the cracks are known to be surface breaking at the backwall.

The above limitations in conventional ultrasonic scanning can prevent an ultrasonic scan operator from accurately measuring the size of a defect while working in the field. As even the smallest of cracks can be of critical importance, the ability to accurately measure small defects would allow more informed decision-making when assessing the safety of pipeline girth welds. This would allow non-critical defects to be left in place and prevent expensive reworking, but would also ensure that truly hazardous defects are identified and that appropriate action is taken. As will be appreciated, improvements in ultrasonic scanning techniques are desired.

It is therefore an object of the present invention to provide a novel ultrasonic scanning system and a novel method of enhancing an ultrasound image.

### SUMMARY OF THE INVENTION

Accordingly, in one aspect there is provided an ultrasonic signal processing method comprising:

deconvolving the received ultrasonic signal to yield a filtered signal;

determining autoregressive extrapolation parameters based on frequency amplitude fluctuations of the filtered signal within a frequency range over which a corresponding reference signal has a high signal-to-noise ratio; and

carrying out an autoregressive spectral extrapolation of the filtered signal using the autoregressive extrapolation parameters to yield an enhanced ultrasonic signal.

According to another aspect there is provided an ultrasonic scanning system comprising:

a processor;

a transmitting probe in communication with the processor, the transmitting probe being configured for emitting an ultrasonic signal into a specimen in accordance with instructions from the processor; and

a receiving probe in communication with the processor, the receiving probe being configured for receiving a return ultrasonic signal from the specimen and for communicating the received ultrasonic signal to the processor, wherein the processor is configured to:
transform the received ultrasonic signal into a frequency domain;
deconvolve the received ultrasonic signal by Wiener filtering to yield a filtered signal;
determine autoregressive extrapolation parameters based on frequency amplitude fluctuations of the filtered signal within a frequency range over which a corresponding reference signal has a high signal-to-noise ratio; and
carry out an autoregressive spectral extrapolation of the filtered signal using the autoregressive extrapolation parameters to yield an enhanced ultrasonic signal.

According to yet another aspect there is provided an ultrasonic measurement system comprising:

a processor; and

an ultrasonic probe in communication with the processor, the ultrasonic probe being configured for generating a transmitted ultrasonic signal in accordance with instructions from the processor, for receiving a return ultrasonic signal and for communicating the received ultrasonic signal to the processor;

the processor being configured for enhancing the received ultrasonic signal by:
transforming the received ultrasonic signal into a frequency domain;
deconvolving the received ultrasonic signal by Wiener filtering to yield a filtered signal;
determining autoregressive extrapolation parameters based on frequency amplitude fluctuations of the filtered signal within a frequency range over which a corresponding reference signal has a high signal-to-noise ratio; and
carrying out an autoregressive spectral extrapolation of the filtered signal using the autoregressive extrapolation parameters to yield an enhanced ultrasonic signal.

According to yet another aspect there is provided an ultrasonic measurement system comprising:

a processor;

an ultrasonic pulser in communication with the processor;

a transmitting probe in communication with the pulser for generating a transmitted ultrasonic signal in response to the pulser;

a receiving probe for receiving a return ultrasonic signal;

a received signal preprocessor in communication with the processor and the receiving probe, the preprocessor comprising an analog-digital converter for digitizing the received ultrasonic signal communicated by the receiving probe, the preprocessor communicating the digitized received ultrasonic signal to the processor, the processor enhancing the digitized received ultrasonic signal by:
transforming the digitized received ultrasonic signal into a frequency domain;
deconvolving the digitized received ultrasonic signal by Wiener filtering to yield a filtered signal;
determining autoregressive extrapolation parameters based on frequency amplitude fluctuations of the filtered signal within a frequency range over which a corresponding reference signal has a high signal-to-noise ratio; and
carrying out an autoregressive spectral extrapolation of the filtered signal using the autoregressive extrapolation parameters to yield an enhanced ultrasonic signal.

According to yet another aspect there is provided an ultrasonic measurement system comprising:

a processor;

an ultrasonic pulser in communication with the processor;

an ultrasonic probe in communication with the pulser, the ultrasonic probe being configured for transmitting an ultrasonic signal and for receiving a transmitted ultrasonic signal;

a received signal preprocessor in communication with the processor and the ultrasonic probe, the preprocessor comprising an analog-digital converter for digitizing a received ultrasonic signal communicated by the receiving probe, the preprocessor communicating a digitized received ultrasonic signal to the processor, the processor enhancing the digitized received ultrasonic signal by:
transforming the digitized received ultrasonic signal into a frequency domain;
deconvolving the digitized received ultrasonic signal by Wiener filtering to yield a filtered signal;
determining autoregressive extrapolation parameters based on frequency amplitude fluctuations of the filtered signal within a frequency range over which a corresponding reference signal has a high signal-to-noise ratio; and
carrying out an autoregressive spectral extrapolation of the filtered signal using the autoregressive extrapolation parameters to yield an enhanced ultrasonic signal.

According to still yet another aspect there is provided a method of enhancing an ultrasonic signal, the method comprising:

receiving a first signal from a specimen; receiving a second signal from a specimen, the second signal being a reference signal, the first and second signals being frequency domain ultrasonic signals;

deconvolving the first signal to yield a filtered first signal; determining autoregressive extrapolation parameters based on frequency amplitude fluctuations of the filtered first signal within a frequency range over which the second signal has a high signal-to-noise ratio; and

carrying out an autoregressive spectral extrapolation of the filtered first signal using the autoregressive extrapolation parameters to yield an enhanced first signal.

### BRIEF DESCRIPTION OF THE DRAWINGS

Embodiments will now be described more fully with reference to the accompanying drawings in which:

FIG. 1 is a schematic diagram, partially in section, of a prior art time of flight diffraction ultrasonic scanning configuration for scanning a pipeline section including a girth weld;

FIG. 2 is a graphical plot of an ultrasonic signal obtained using the ultrasonic scanning configuration of FIG. 1;

FIG. 3 is a time of flight diffraction image of the girth weld as a result of a circumferential pipeline section ultrasonic scan using the ultrasonic scanning configuration of FIG. 1;

FIG. 4 is a schematic diagram of an embodiment of the subject ultrasonic scanning system;

FIG. 5 is a flow chart of an ultrasound image enhancement method employed by the ultrasonic scanning system of FIG. 4;

FIG. 6 is a flow chart of an autoregressive parameter selection procedure performed during the method of FIG. 5;

FIG. 7 is a graphical plot of a system response curve superimposed over a Wiener filtered signal, obtained using the method of FIG. 5;

FIGS. 8a and 8b are graphical plots of a final best-fit curve, and an extended final best-fit curve showing ranges of possible frequency window boundary values m and n, respectively, superimposed over a Wiener filtered signal, obtained using the method of FIG. 5;

FIG. 9 is another embodiment of the subject ultrasonic scanning system;

FIGS. 10a to 10c are graphical plots of a simulated ultrasonic signal, an autoregressive (“AR”) extrapolation result (p=20) obtained using the method of FIG. 5, and an analytic signal magnitude of the AR extrapolated signal, respectively, in accordance with Example 1 disclosed herein;

FIG. 11 is a graphical plot of a reference ultrasonic signal echo used with Example 2 disclosed herein;

FIGS. 12a to 12e are graphical plots of a measured ultrasonic signal, a Wiener filtered signal, a single AR order extrapolation result (p=15) obtained using the method of FIG. 5, a ranged AR order extrapolation result (pₘᵢₙ=10, pₘₐₓ=15) obtained using the method of FIG. 5, and an analytic signal magnitude of the ranged AR extrapolation, respectively, in accordance with Example 2 disclosed herein;

FIG. 13 is a graphical plot of an analytic signal magnitude of a processed ultrasonic signal for a 3.14 mm thick aluminum plate (normal incidence, 7.5 MHz probe frequency, 100 MHz sampling frequency), in accordance with Example 3 disclosed herein;

FIGS. 14a to 14e are graphical plots of a measured ultrasonic scan of a 0.82 mm thick aluminum plate in an immersion tank (normal incidence, 7.5 MHz probe frequency, 100 MHz sampling frequency), a Wiener filtered signal, a single AR order extrapolation result obtained using the method of FIG. 5, a ranged AR order extrapolation result obtained using the method of FIG. 5, and an analytic signal magnitude of the single AR order extrapolation result, respectively, in accordance with Example 3 disclosed herein.

FIGS. 15a to 15d are graphical plots of a measured ultrasonic scan of a 0.42 mm thick stainless steel plate in an immersion tank (normal incidence, 7.5 MHz probe frequency, 100 MHz sampling frequency), an analytic signal magnitude of the measured signal, a ranged AR order extrapolation result obtained using the method of FIG. 5, and an analytic signal magnitude of the ranged AR order extrapolation result, respectively, in accordance with Example 3 disclosed herein;

FIG. 16 is schematic diagram of an immersion tank configuration of a double flat bottomed hole specimen, used in accordance with Example 4 disclosed herein;

FIGS. 17a to 17c are graphical plots of a measured ultrasonic scan of a specimen having two flat bottomed holes with a depth difference of 0.2794 mm (immersion tank, 7.5 MHz probe, sample frequency 500 MHz), a ranged AR order extrapolation result obtained using the method of FIG. 5, and an analytic signal magnitude of the ranged AR order extrapolation result, respectively, in accordance with Example 4 disclosed herein;

FIG. 18a is a schematic diagram of a cracked bar specimen, and FIG. 18b is a photograph of a fracture surface of an oxidized cracked bar specimen, used in accordance with Example 5 disclosed herein;

FIG. 19 is a photograph of a calibration block, used in accordance with Example 5 disclosed herein;

FIG. 20 is an S-scan image of a measured ultrasonic scan reference echo, in accordance with Example 5 disclosed herein; and

FIGS. 21a and 21b are S-scan images of a measured ultrasonic scan of a cracked bar specimen, and a ranged AR order extrapolation result obtained using the method of FIG. 5, respectively, in accordance with Example 5 disclosed herein.

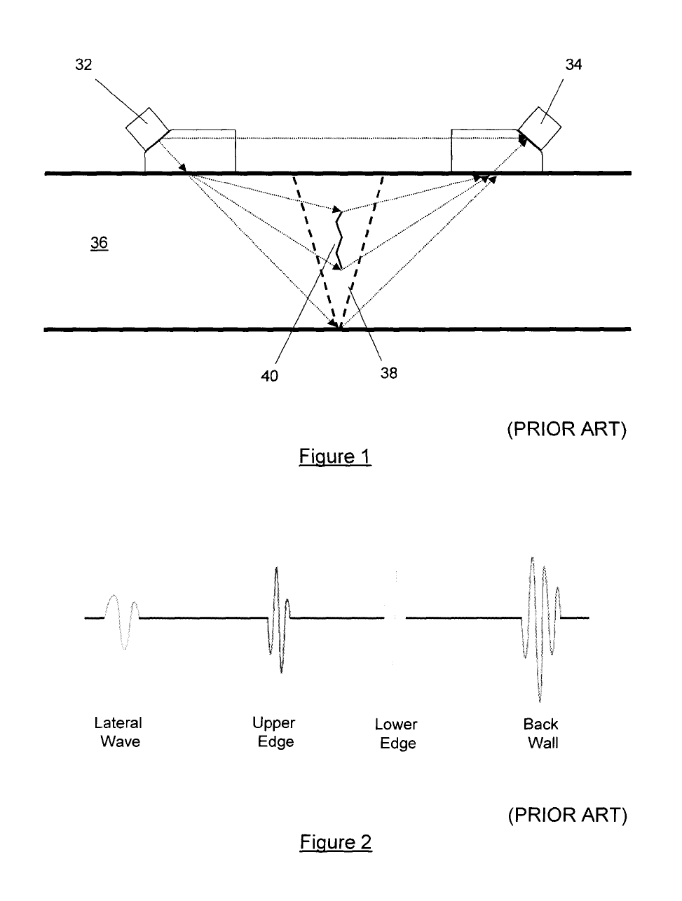

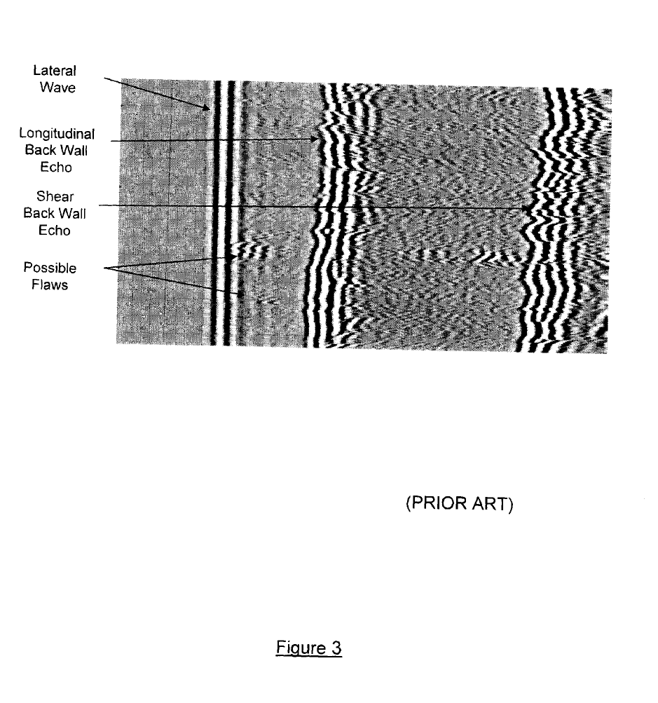

### DETAILED DESCRIPTION OF THE EMBODIMENTS

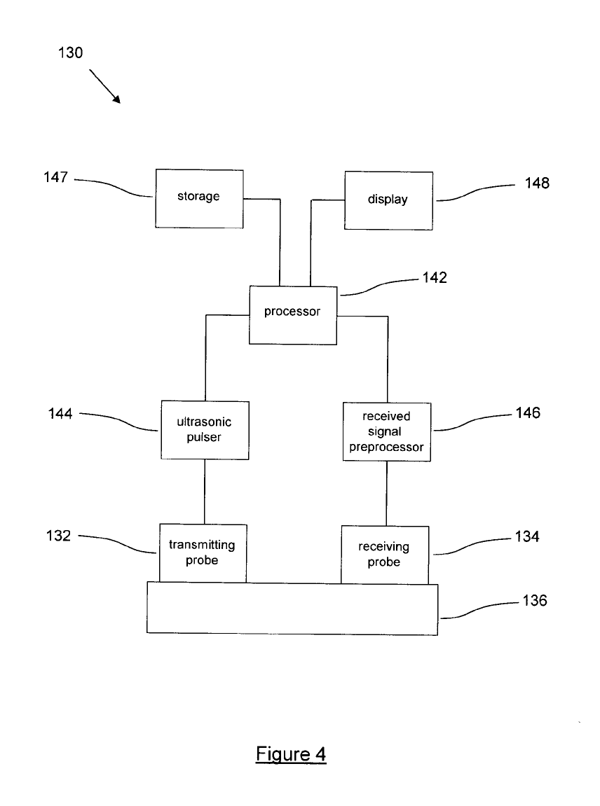

Turning now to FIG. 4, an ultrasonic scanning system is shown and is generally indicated by reference numeral 130. Ultrasonic scanning system 130 allows structural materials to be ultrasonically scanned for the purpose of non-destructive testing and, in the embodiment shown, is used for the ultrasonic scanning of girth welds joining adjacent oil and gas pipeline sections for the purpose of defect detection.

As can be seen, ultrasonic scanning system 130 comprises a processor 142 that is in communication with an ultrasonic pulser 144, which is itself in communication with an ultrasonic transmitting probe 132. Pulser 144 is configured to generate an ultrasonic waveform having characteristics in accordance with instructions received from processor 142, and to communicate the ultrasonic waveform to the transmitting probe 132 which in turn generates and broadcasts a corresponding ultrasonic signal. Processor 142 is also in communication with a received signal preprocessor 146, which is itself in communication with an ultrasonic receiving probe 134. Although not shown, the received signal preprocessor 146 comprises an amplifier, a filter, and an analog-to-digital converter. Received signal preprocessor 146 pre-processes received ultrasonic signal echoes to convert the received ultrasonic signal echoes into digital form and communicates the digitized received ultrasonic signal echoes to processor 142. Processor 142 is also in communication with storage 147 and display 148. Processor 142 can communicate the digitized received ultrasonic signal echoes to storage 147 allowing the stored digitized received ultrasonic signal echoes to be later retrieved by processor 142, as desired or as required. Processor 142 can also communicate the digitized received ultrasonic signal echoes to display 148 allowing an ultrasound image to be displayed in graphical form. Processor 142 can also enhance the digitized received ultrasound signal echoes through further signal processing, as will be described below, allowing an enhanced ultrasonic image to be generated. As will be appreciated, if the processor 142 is conditioned to enhance the ultrasonic signal echoes, the enhanced ultrasonic signal echoes can similarly be sent to storage 147 and display 148.

During use, the transmitting probe 132 and receiving probe 134 are initially positioned proximate the specimen 136 (in this case the pipeline section) to be scanned. With the probes 132 and 134 properly positioned the processor 142 in response to operator input provides instructions to the pulser 144 causing the pulser 144 to generate an ultrasonic waveform. The ultrasonic waveform generated by the pulser 144 is then applied to the transmitting probe 132 causing the transmitting probe to broadcast an ultrasonic signal into the specimen 136. Reflections or echoes of the ultrasonic signal are picked up by the receiving probe 134 and are conveyed to the received signal preprocessor 146 for preprocessing. Once preprocessed, the digitized received ultrasonic signal echoes are conveyed to the processor 142.

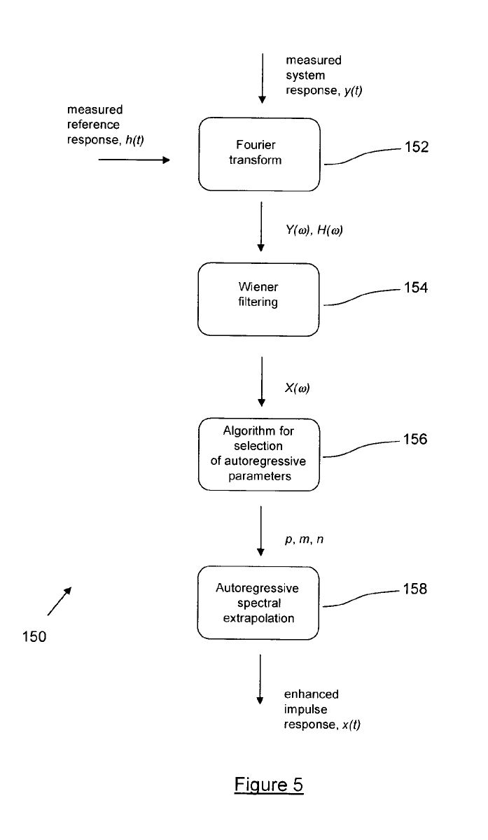

Typically during scanning, the transmitting and receiving probes 132 and 134 are moved circumferentially about the pipeline section allowing an image of the complete circumferential volume of the pipeline section to be generated. The processor 142, as mentioned above, can store and/or display the received ultrasonic signal echoes as received by the receiving probe 134 or can further process the received ultrasonic signal echoes to allow an enhanced ultrasound image to be generated. The steps performed by the processor 142 during this further processing are shown in FIG. 5 and generally identified by reference numeral 150.

The further processing methodology comprises two major components, namely filtering and deconvolution and autoregressive spectral extrapolation. Deconvolution allows the components of the received ultrasonic signal echoes that originate from features of the ultrasonic scanning system 130 itself to be removed from the received ultrasonic signal echoes. This may have the effect of narrowing the time domain duration of the received ultrasonic signal echoes while improving the temporal resolution. To do this, an ultrasonic signal is broadcast into a material that is free of defects and the received return ultrasonic signal representing the reference system response is stored and is used thereafter as a reference signal.

In order to perform the deconvolution, it is assumed that the impulse response x(t) of the specimen 136 is a discrete sum of delta functions corresponding to individual reflectors inside the specimen. In practice, the system response y(t) is a convolution of impulse response x(t) with the reference system response h(t). Reference system response h(t) incorporates the effects of, for example, transducers within the transmitting and receiving probes 132 and 134, any coupling fluid applied between transmitting and receiving probes 132 and 134 and the specimen 136, and any cables connecting the pulser 144 to transmitting probe 132 and connecting the receiving probe 134 to received signal preprocessor 146. Reference system response h(t) also incorporates the effect of the bulk material of specimen 136, however, for the measurement of reference system response h(t), a defect-free region of any material may be used. The received ultrasonic signal echoes will also contain some noise, n(t). Accordingly, the system response y(t) can be described by Equation (1) below as:

y(t)=h(t)*x(t)+n(t) (1)

where * is the linear convolution operator.

As shown in FIG. 5, during deconvolving step 152 the received ultrasonic signal representing the system response y(t) and the reference signal representing the reference system response h(t) are subjected to a Fourier transform. In the frequency domain, Equation (1) becomes:

Y(ω)=H(ω)X(ω)+N(ω) (2)

At step 154, the Fourier transformed system responses are deconvolved using Wiener filtering. Wiener filtering is based on the minimization of a least square error, as described in “Deconvolution of Images and Spectra”, 2^{nd} edition, Academic Press, Toronto, 1997, by Jansson, the content of which is incorporated herein by reference. This deconvolution takes the form:

$$X ( ω ) = \frac{Y ( ω ) {H}^{*} ( ω )}{{\vert H ( ω ) \vert}^{2} + {Q}^{2}} \qquad ( 3 )$$

where H*(ω) is the complex conjugate of H(ω). The value Q² is a noise desensitizing factor selected to ensure that the denominator of Equation (3) never goes to zero. A recommended value for Q² is:

Q²=10⁻²|H(ω)|ₘₐₓ² (4)

as described in “Engineering Applications of Ultrasonic Time-Of-Flight-Diffraction”, 2^{nd} edition, Research Studies Press Ltd., Hertfordshire, 2001, by Charlesworth et al., the content of which is incorporated herein by reference.

Following Wiener filtering, the autoregressive spectral extrapolation steps are performed. The autoregressive process is a time series in which the present output is determined by a linear combination of past outputs. By this definition, autoregressive spectral extrapolation is a signal processing technique that extrapolates the information contained in a signal from the frequencies where the signal-to-noise ratio (“SNR”) is high, to the frequencies where the SNR is low. It is a technique often used to model systems that can be approximated as linearly-combined first and second order responses to harmonic oscillators, as described in both “Time Series Modeling and Maximum Entropy”, Physics of the Earth and Planetary Interiors, 12 (1976) pp. 188-200, by Ulrych et al., and in “A Review of Autoregressive Spectral Extrapolation and Wiener Filtering”, University of Toronto, 2003, by Dusatko et al., the contents of which are incorporated herein by reference.

Autoregressive spectral extrapolation is best performed using a portion of a digitized signal x, that has a high SNR in the frequency domain. That is, the spectrum of the signal in the frequency range where the SNR is high is extrapolated to infer the spectral shape at frequencies both below and above the useful bandwidth of the ultrasonic scanning system. As described in “Improving the time-resolution and signal-to-noise ratio of ultrasonic NDE signals”, Ultrasonics, 41, 2004, by Honavar et al., the content of which is incorporated herein by reference, the extrapolations for the low frequency end (Equation (5) below) and the high frequency end (Equation (6) below) have the following forms:

$$\begin{matrix}{\hat{x}}_{k} = - {\sum }_{i = 1}^{p} {a}_{i}^{*} {x}_{k + i} \\ k = [ m - 1 , m - 2 , … 1 ] & ( 5 ) \\ {\hat{x}}_{q} = - {\sum }_{i = 1}^{p} {a}_{i}^{*} {x}_{q - i} \\ q = [ n + 1 … {N}_{f} ] & ( 6 )\end{matrix}$$

where \hat{x} is an extrapolated value of X(ω), p is the autoregressive order number, (m . . . n) is the frequency window of the digitized signal, N_{f} is the Nyquist frequency, and aᵢ and aᵢ* are autoregressive coefficients obtained using the Burg method and their complex conjugates respectively (also see “Modern Spectral Estimation: Theory and Application, Prentice Hall, Englewood Cliffs, N.J., 1988, by Kay). Specifically, the autoregressive coefficients aᵢ and aᵢ* are obtained by selecting coefficients to minimize the sum of squares of forward and reverse prediction error; the prediction error being minimized is:

$${e}^{2} = \frac{1}{{N}_{f} - p} {\sum }_{j = p + 1}^{{N}_{f}} {\vert X ( j ) - {\sum }_{k = 1}^{p} {a}_{k} X ( j - k ) \vert}^{2} + \frac{1}{{N}_{f} - p} {\sum }_{j = 1}^{{N}_{f} - p} {\vert X ( j ) - {\sum }_{k = 1}^{p} {a}_{k}^{*} X ( j + k ) \vert}^{2} \qquad ( 7 )$$

As can be seen from Equations (5) to (7), the autoregressive extrapolation utilizes three autoregressive parameters, namely the autoregressive (“AR”) order number, p, and boundaries m and n of the frequency window {m . . . n}, within which the SNR is sufficiently high to form a base for the signal extrapolation operation.

The AR order number, p, is the number of previous known points to be used to calculate the next point during an extrapolation. If the AR order number p is set to 20, then to extrapolate beyond the frequency window boundary (n in Equation (6)) the previous twenty points are used in the calculation of \hat{x}ₙ₊₁. These extrapolated points are then used to continue the extrapolation. However, care must be taken in selecting a value for the AR order number p. A lower value for the AR order number p will allow the extrapolation to be performed more quickly, but might not yield as sharp an image as a higher value of the AR order number p. Additionally, if the AR order number p is too low the extrapolation may not improve or sharpen the image significantly over the Wiener filtered result. A larger value for the AR order number p means that more information is used from the raw signal when performing the extrapolation; however, a higher AR order number p reduces computational speed. If the AR order number p is too high, the extrapolation will begin to model the signal noise and spurious spikes will appear in the final extrapolation.

Determining an appropriate value of the AR order number p for a given signal is therefore not straightforward, and using a very high AR order number in a brute force manner does not necessarily produce better results. Ideally, the value of the AR order number p will be large enough to sharpen the ultrasonic signal echoes, but will not be so large that the signal noise begins to be modeled.

A known approach for determining the frequency window boundaries m and n utilizes a fractional drop of the reference signal amplitude, H(ω), to define these parameters. However, this approach is based on the assumption that ultrasonic signal echoes resulting from defects or flaws in the specimen have similar frequency content to the reference signal. This is often not the case, with the result being that the autoregressive extrapolation of the measured signal is not optimized. Despite this concern, the reference signal echo is a logical place to obtain an initial estimate of the frequency window boundaries m and n, since the reference signal is typically a strong signal that is relatively free of noise. However, a more robust examination of the flaw ultrasonic signal echoes should ultimately be performed in order to find the final frequency window to be used to serve as a basis for extrapolation.

If the frequency window {m . . . n} is too wide, then the autoregressive extrapolation may not adequately improve the flaw image beyond that of the Wiener filtered signal, X(ω). However, if too narrow a frequency window is selected, very little information is retained during the extrapolation phase, and thus useful data is lost. Interestingly, it has been found that symmetry of the spectrum within the frequency window plays an important role when performing the extrapolation. If the extrapolation is carried out based on a frequency window for which the signal amplitudes at frequencies m and n differ significantly, then the technique yields unpredictable results.

Additionally, the selection of the frequency window boundaries m and n affects the choice of the AR order number p. For instance, a selection of a wide frequency window {m . . . n} but a low value of the AR order number p is likely to yield a poor extrapolation, because the extrapolation will be conducted with only a small portion of the information contained in the selected frequency window. This indicates that the AR order number and frequency window boundary values p, m, and n are interrelated.

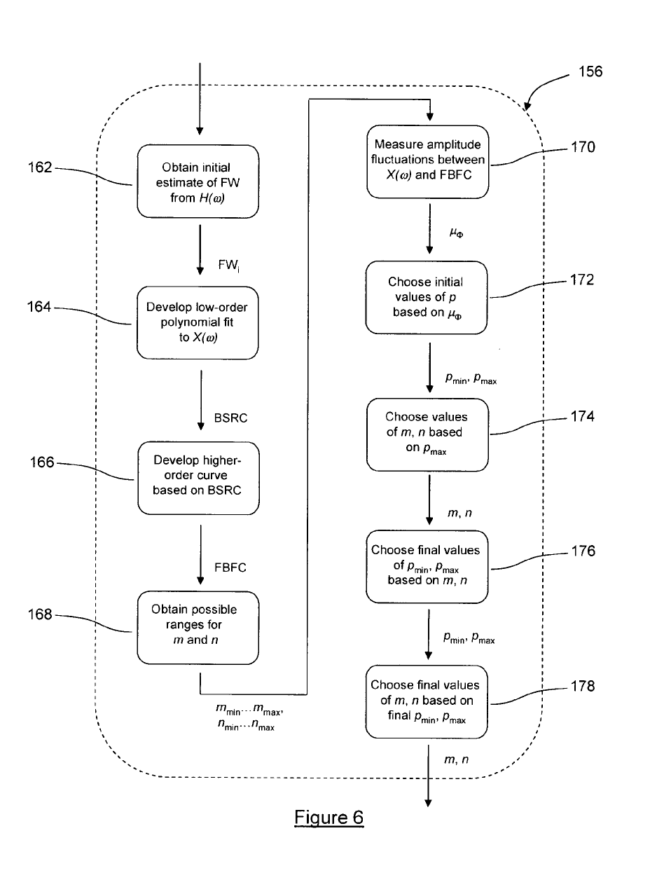

Returning back to FIG. 5, at step 156 following Wiener filtering, the AR order number and frequency window boundary values p, m, and n are selected based on both the Wiener filtered signal, X(ω), and the reference signal H(ω) are selected. The steps performed in order to select the AR order number and frequency window boundary values are shown in FIG. 6. During this process, the reference signal is used to get an initial estimate for the frequency window, FW, (step 162). A low-order polynomial approximation to the Wiener filtered signal X(ω) based on FW, is then developed (step 164). The resultant curve obtained in step 164 is analyzed to produce a higher-order polynomial approximation “best-fit curve” (step 166). Narrow ranges of possible values for the frequency window boundary parameters m and n are then obtained (step 168). A quantitative measure of the frequency domain amplitude fluctuations within FW, is then determined (step 170). Initial values of the AR order number p based on the amplitude fluctuations are chosen (step 172) and the initial values of the AR order number p are used to choose initial values for the frequency window boundaries m and n (step 174). The width of the frequency window determined by the initial boundary values m and n are then examined and the final AR order number values p are determined. The final AR order number values p are then used to determine final frequency window boundary values m and n.

At step 162, an initial frequency window of the Wiener-filtered signal, X(ω), that is of interest, namely that where the SNR is relatively high is established. The Fourier transform of the reference signal, namely H(w), obtained in step 154, is utilized to obtain an initial estimate for the frequency window, since the reference echo is a low-noise signal that is easily characterized. The initial estimate of the frequency window, FWᵢ, is the full-width, half-maximum (“FWHM”) of the reference echo spectrum H(w).

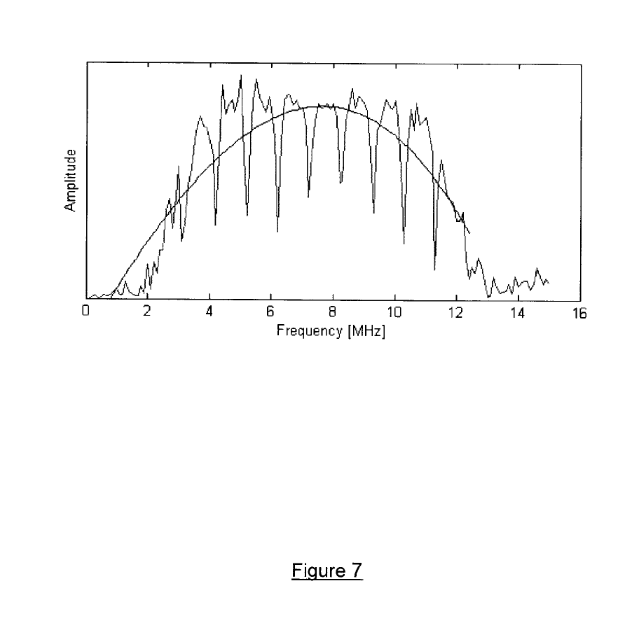

At step 164 a third-order polynomial approximation to the Weiner-filtered echo signal, X(ω) within the frequency window FW, is generated. Here, any of a variety of known curve fitting techniques may be applied. Such an approximation is depicted in FIG. 7, and is termed the basic system response curve (“BSRC”). The BSRC is a smooth approximation having a profile that is similar to that of the reference echo, H(ω), (i.e. a Gaussian peak) but which tends to peak near the centre frequency of the echo signal, X(ω), which may differ substantially from the peak of H(ω). For this reason, the BSRC provides a better basis than H(ω) for finding suitable frequency window boundary values m and n.

As is known in the art, the fit at the end points of a polynomial curve is often poor, and the end sections of the fitted curve can be distorted. For this reason, the frequency window used for the fitting of BSRC is extended beyond FW, so that the curve will better model the signal within FW. It has been found empirically that extending each side of the frequency window by half the number of initial points in FW, produces good results for all types of signals tested.

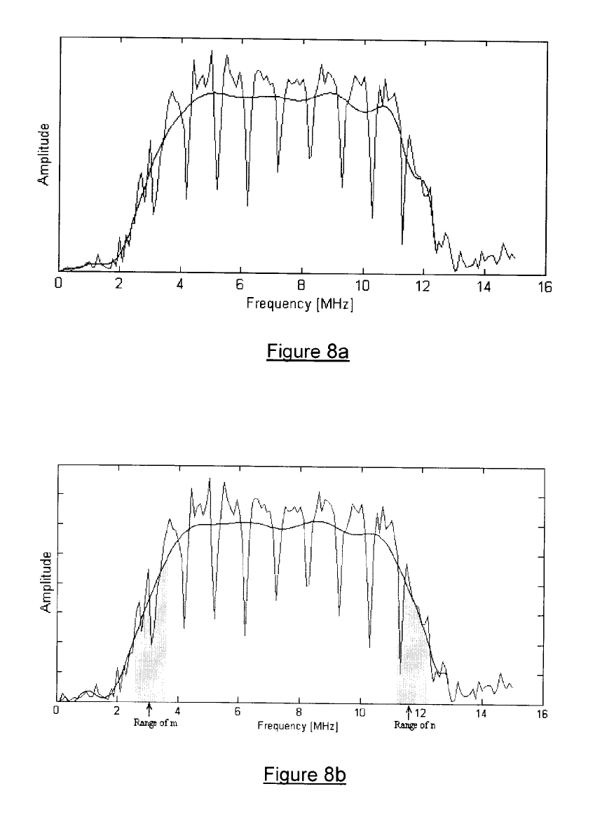

At step 166 the final best-fit curve (“FBFC”) from the BSRC is determined. The boundary points of the FWHM of the BSRC are first determined. This FWHM window is then extended on both sides by twice the number of points in the FWHM window to generate a higher-order polynomial fit to X(ω). Such an extension to the FWHM window width is required to ensure that the higher-order polynomial fit represents a good approximation to X(ω) at the edges of the FWHM window. This scheme for extending the FWHM window width was found empirically to yield good results for all types of signals tested. It was also found empirically that the polynomial degree, z, yielding the best fit was one-fifth of the number of points in the extended FWHM window, with the limitation that z be bounded by the range {5 . . . 20}. By this approach, the FBFC was found to follow major signal features, but not to be overly-susceptible to noise spikes, as based on signal data obtained experimentally using ultrasound probe frequencies ranging from 2.25 MHz to 10 MHz. FIG. 8a shows an example of an FBFC fitted to X(ω), based on the BSRC plotted in FIG. 7.

At step 168 the possible ranges for the frequency window boundary values m and n are obtained from the FBFC. Here, the FBFC is analyzed to determine a frequency range of the Wiener filtered signal, X(ω), that is to be retained and utilized for extrapolation to other frequencies of X(ω) where the SNR is low. If necessary, the frequency range of the FBFC can be first extended, and the FBFC recalculated to ensure that it includes all frequencies for which there is significant signal strength of X(ω). Honarvar et al. have shown that this region should include any frequencies for which the echo strength (i.e. X(ω)) is within 8 dB of its strength at the peak frequency.

At this point, a final high-order polynomial best-fit curve (FBFC) is obtained for the Wiener filtered signal, X(ω), extending on either side of the peak frequency over a relatively wide window for which there is any significant signal strength, as depicted in FIG. 8b. The corresponding frequency indices of this frequency window may be defined as mₘᵢₙ and nₘₐₓ, according to the 8 dB criterion described above, and represent the largest possible frequency window width to be used in the extrapolation process.

Values for boundary values mₘₐₓ and nₘᵢₙ can then be selected as the frequencies at which there is a 2 dB drop of the FBFC from its peak value. This is different from the value of 3 dB suggested by Honarvar et al.; however it has been found empirically that the 2 dB value is effective for specifying the smallest potential frequency window width to be used for extrapolation. The resulting possible ranges of frequency window boundary values m and n, {mₘᵢₙ . . . mₘₐₓ, nₘᵢₙ . . . nₘₐₓ} are depicted in FIG. 8b.

The “8 dB” and “2 dB” criteria defined above is referred to herein as a “first set of criteria”. However, as will be appreciated, other criteria may be used to define the “first set of criteria”.

At step 170 frequency amplitude fluctuations between X(ω) and the FBFC are measured. For each frequency index i between frequency window boundary values mₘᵢₙ and nₘₐₓ, the positive difference between the amplitudes of FBFC and X(ω) is divided by the amplitude of the FBFC at that frequency, as expressed by Equation (8) below:

$${Φ}_{i} = \frac{\vert \vert X ( ω ) \vert - \vert {FBFC}_{i} \vert \vert}{\vert {FBFC}_{i} \vert} \qquad ( 8 )$$

Their sum, ΣΦ, can readily be determined over the entire curve. An average of all the elements may be defined as:

$${μ}_{Φ} = \frac{\sum {Φ}_{i}}{( {n}_{high} - {m}_{low} )} \qquad ( 9 )$$

where μ_{Φ} is herein termed the “Average Best Fit-to-Difference Ratio”.

At step 172 an initial value of the autoregressive order number, p, based on ratio μ_{Φ} is determined A lower value of ratio μ_{Φ} generally reflects a smoother X(ω) profile (because the FBFC is following X(ω) more closely), and suggests that a lower value of the AR order numberp would be appropriate. Dusatko et al. suggest an approach in which a range of values for the AR order number p is considered (i.e. pₘᵢₙ . . . pₘₐₓ), but provide no indication as to how this range should be selected. Based on empirical studies of the relationship between ratio μ_{Φ}, values of the AR order number p, and the quality of extrapolated spectra achieved for many signals obtained with ultrasound probes ranging from 2.25 MHz to 10 MHz, the following ranges of possible values for the AR order number p (pₘᵢₙ, pₘₐₓ) were determined as set out in Table 1 below:

TABLE 1

| Average Best | | |
|---|---|---|
| Fit-to-Difference | Min AR Order | Max AR Order |
| Ratio μ_{Φ} | pₘᵢₙ | pₘₐₓ |
| μ_{Φ} ≦ 0.1 | 10 | 15 |
| 0.1 < μ_{Φ} ≦ 0.125 | 11 | 16 |
| 0.125 < μ_{Φ} ≦ 0.15 | 12 | 17 |
| 0.15 < μ_{Φ} ≦ 0.175 | 13 | 18 |
| 0.175 < μ_{Φ} ≦ 0.2 | 14 | 19 |
| μ_{Φ} ≧ 0.2 | 15 | 20 |

At step 174 initial values of the frequency window boundaries m and n are identified. For each boundary index m and n lying within its respective range, m_{low} . . . m_{high} and m_{low} . . . n_{high}, as determined in step 168, an average value of μ is calculated for the pₘₐₓ points that would be used in an autoregressive extrapolation from that given point. This average value for each point in the ranges m_{low} . . . m_{high} and n_{low} . . . n_{high} can be expressed according to:

$${μ}_{LS , i} = \frac{{\sum }_{j = i}^{i + p - 1} {Φ}_{j}}{p} , \\ i = {m}_{low} … {m}_{high} \\ {μ}_{RS , i} = \frac{{\sum }_{j = ( i - p + 1 )}^{i} {Φ}_{j}}{p} , \\ i = {n}_{low} … {n}_{high} \qquad ( 10 )$$

where the subscripts LS and RS refer to the left (i.e. low frequency) side and right (i.e. high frequency) side of the FBFC, respectively, and p corresponds to pₘₐₓ.

Spectral extrapolation is known to work best when there is approximate symmetry between the two sides of the frequency window under consideration. To this end, the elements of {m_{low} . . . m_{high}} are paired with the elements of {n_{low} . . . n_{high}} that are the closest matches in terms of spectral amplitude, i.e. X(ω). The pair of elements that correspond to the lowest values of μ_{LS} and μ_{RS}, as calculated using Equation (10), is then considered to represent the most suitable frequency window and these elements are used as the initial frequency window boundary values m and n.

At step 176 the final values of the AR order number p are chosen. The width of the frequency window (i.e. n−m), as determined in step 174, is examined according to a set of criteria to determine if the range of the AR order number values p chosen in step 172 is appropriate. Firstly, the value of pₘₐₓ cannot be greater than the number of points in the frequency window (n−m+1). This is because in an autoregressive extrapolation technique, the number of points used to determine the next extrapolated point cannot exceed the number of points in the original data set that is being extrapolated. Should pₘₐₓ exceed this number then it is reduced to the number of points in the frequency window, according to:

IF pₘₐₓ>(n−m+1)

THEN pₘₐₓ=(n−m+1)

Secondly, the extrapolation will not perform well if the value of pₘₐₓ is less than half the number of points in the frequency window, as this would imply that only a small fraction of the information contained within the data set being extrapolated is being used for the extrapolation process. As such, if pₘₐₓ is less than half the number of points in the frequency window, then pₘₐₓ is increased to equal half the number of points in the FW:

$$IF {p}_{max} < \frac{( n - m + 1 )}{2} \\ Then {p}_{max} = \frac{( n - m + 1 )}{2} \qquad ( 12 )$$

where pₘₐₓ is an integer.

It has also been found through experimentation that values of that are much lower than pₘₐₓ are not best suited for the extrapolation process. The procedure can therefore be simplified by eliminating these very low values from further consideration according to:

pₘᵢₙ=pₘₐₓ−10 (13)

where pₘₐₓ and pₘᵢₙ are integers. These values of pₘᵢₙ and pₘₐₓ are the final values of the AR order number p. At step 178, the final values of frequency window boundaries m and n are chosen using the routine described with reference to step 174, utilizing the final value of pₘₐₓ determined in step 176.

The criteria defined in Equations (11), (12), and (13) is referred to herein as a “second set of criteria”. However, as will be appreciated, other criteria may be used to define the “second set of criteria”.

Following determination of the autoregressive parameters p, m and n at step 156, the autoregressive spectral extrapolation is carried out using the established autoregressive parameters p, m and n. One of two techniques may be used for this extrapolation. The extrapolation may be performed using pₘₐₓ and the final values of the frequency window values m and n. Alternatively, the extrapolation may be performed multiple times using a range of p values lying in the range {pₘᵢₙ . . . pₘₐₓ} and then averaged to produce a single extrapolated signal, as described by Dusatko et al. In this technique, the final values of the frequency window boundaries m and n that are used for the values of p within {pₘᵢₙ . . . pₘₐₓ}, are determined in step 178 using pₘₐₓ. Any number of values of p may be used.

As will be appreciated, the methodology described above allows the autoregressive parameters utilized for autoregressive spectral extrapolation to be determined in an automatic manner, thereby reducing the requirement for the operator to interpret or input data in order to carry out the autoregressive spectral extrapolation.

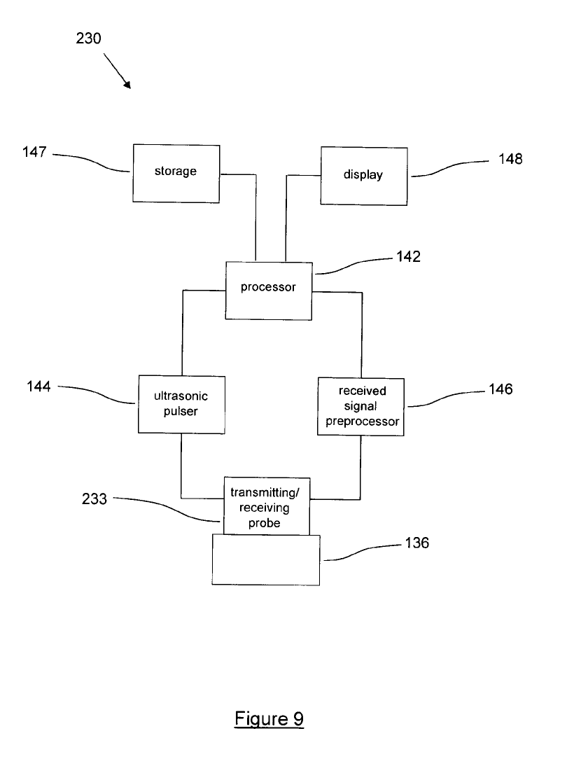

FIG. 9 shows another embodiment of an ultrasonic scanning system, generally indicated by reference numeral 230. The ultrasonic scanning system 230 is very similar to that of the previous embodiment with the exception that instead of using separate transmitting and receiving probes, a single transmitting/receiving (i.e. transceiver) probe 233 that communicates with both the processor 144 and received signal preprocessor 146 is employed.

Although the above embodiments show the processor 142 in communication with the pulser 144, which in turn is in communication with an ultrasonic probe, the pulser may be integrated with either the processor or the ultrasonic probe. Similarly, although the received signal preprocessor 146 is shown in communication with both an ultrasonic probe and the processor 142, the preprocessor may be integrated with either the processor or the ultrasonic probe.

The transmitting probe, receiving probe, transmitting/receiving probe, and ultrasonic probe of the embodiments described above may be any of a phased array probe and a “single-element” probe, as is known in the art, or may be any other form of ultrasonic probe known in the art. As will be appreciated, the processing methodology described above to generate an enhanced ultrasound image is suitable for use in a variety of ultrasonic phased array scanning equipment and similar devices such as for example those manufactured by Olympus NDT of Waltham, Mass., GE Sensing & Inspection Technologies of Leviston, Pa., Sonatest Ltd of Milton Keynes, U.K. and Zetec of Snoqualmie, Wash. to name specifically a few.

Further processing to enhance ultrasonic signal echoes can be performed in real time as the signal echoes are received, or can be preformed as post-processing on previously stored signal echoes. Although the further processing is described as employing Wiener filtering, those of skill in the art will appreciate that any suitable deconvolution method may be used to deconvolve the received ultrasonic signal echoes.

Although the above ultrasonic scanning are described with reference to systems defect detection in welds within the walls of an oil or gas pipeline, as will be appreciated, the ultrasonic scanning systems are not limited to this use and may in fact be used for the ultrasonic scanning of other specimens, such as structural material that is used in the field of nuclear energy, petroleum exploration, rail transportation, air transportation, and the like.

The following examples illustrate various applications of the above-described systems and methods.

### Example 1

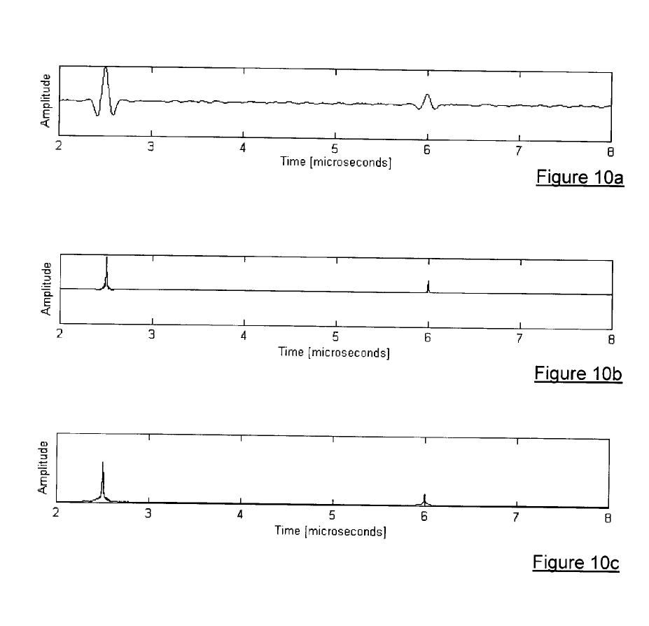

In this example, a simulated A-Scan was developed using idealized echo response functions, linear convolution and simulated noise. The goal of this experiment was to have the sinusoidal echoes of the original simulated signal (shown in FIG. 10a) become much sharper “spikes” after signal processing. As an example, the result using a single autoregressive extrapolation with p=20 is shown in FIG. 10b. The result obtained by using a range of p-values did not substantially differ from the result shown in FIG. 10b. The further signal processing described above performed as required, such that the signal echoes were substantially sharpened after processing.

### Example 2

In this example, a simple hole of 0.81 mm in diameter was drilled into an aluminum specimen. The depth of the hole came to within 9.67 mm of the opposing surface. An ultrasonic probe was placed on this opposing surface in order to detect the bottom of the hole. The goal of this experiment was to test the signal processing on a measured signal. With an 8.9 mm thick delay line on a 10 MHz probe, the echo from the hole was expected at 10.761 μs after the excitation of the original ultrasonic pulse. The values for the extrapolation parameters were determined as follows: m=2.5 MHz, n=9 MHz, pₘᵢₙ=10, pₘₐₓ=15.

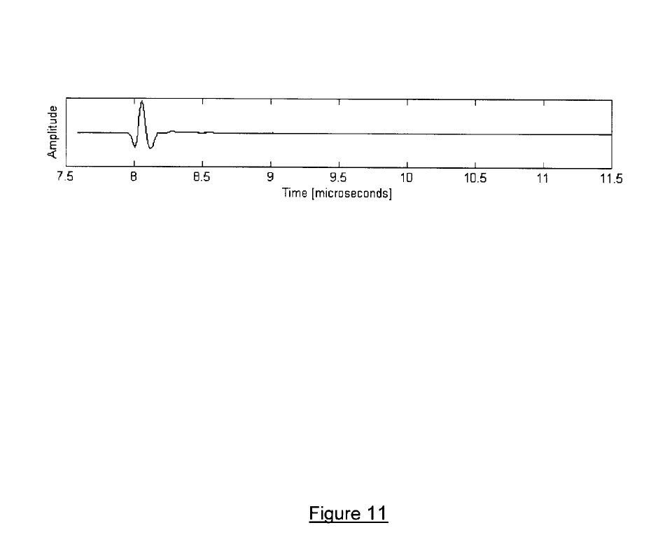

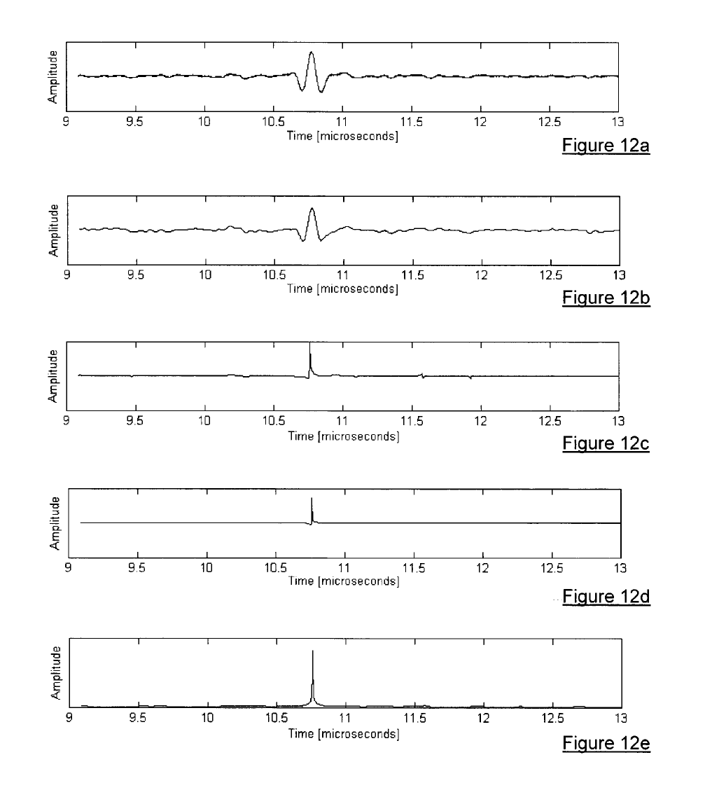

The reference echo was taken from the echo that occurred in the delay line, as shown in FIG. 11. The original echo signal and its Weiner-filtered counterpart are shown in FIGS. 12a and 12b, respectively. After processing, the image was sharpened to the time index 10.764 μs for both a single value of p, and a range of p, as shown in FIGS. 12c and 12d, respectively. As can be seen, the results achieved using a range of values for p proved to exhibit less noise than those obtained with a single value of p. FIG. 12e shows an analytic signal magnitude of the ranged AR extrapolation result plotted in FIG. 12d.

### Example 3

An ultrasonic beam directed at a flat plate at normal incidence will produce multiple back and front wall echoes as the wave continually reflects between the faces of the specimen. The distance between each echo is known because the roundtrip travel time of sound corresponds to twice the thickness of the plate. In this example, a probe was placed in an immersion tank and was used to examine several different thin plates at normal incidence to obtain ultrasonic signals with multiple closely-spaced echoes. A reference signal was obtained by using a 12.36 mm thick Plexiglas plate from which a single, well-defined echo was obtained.

The goal of this experiment was to produce and analyze signals with multiple echoes. By reducing the thicknesses of the specimen, the multiple ultrasonic echoes will become closer together in the time domain. Eventually these echoes will begin to overlap in time. This experiment was designed to test the ability to resolve such overlapping echoes. In the tabulated results, the calculated thickness was taken as the average value determined from time differences between the first four echoes in the ultrasonic signal, shown in FIG. 13. The first four echoes were chosen to be representative; beyond this number the echoes were not always clearly defined.

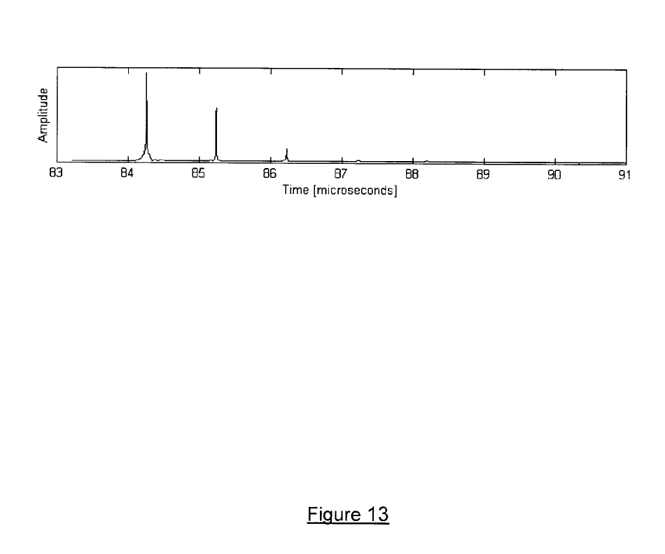

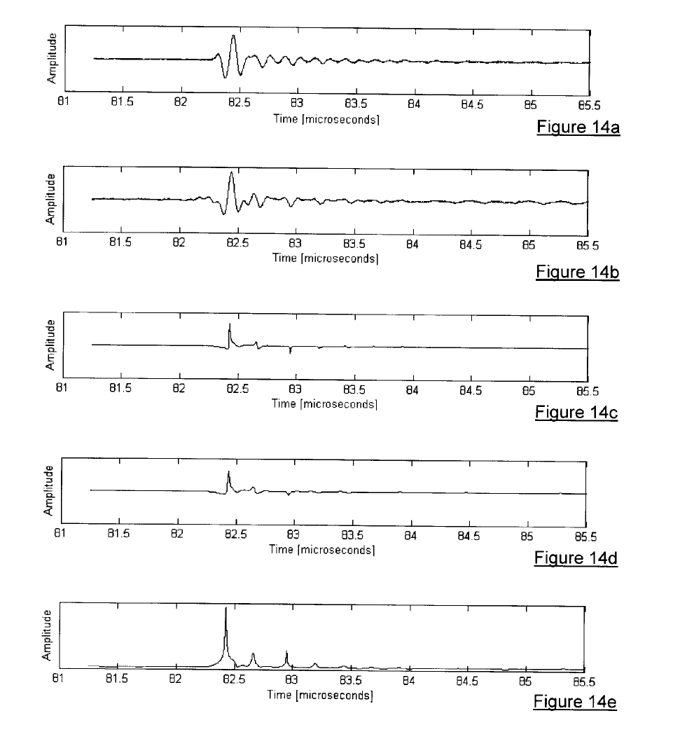

The first experiment was conducted with a 7.5 MHz probe with a sampling frequency of 100 MHz. The results are summarized in Table 2 below. The autoregressive extrapolation results using a range of p values showed a slight improvement over those achieved using a single value of p. However, the observed difference was marginal, and both techniques resulted in estimates of plate thickness that were accurate to within hundredths of a millimeter. FIGS. 14a to 14e are graphical plots of a measured ultrasonic scan of a 0.82 mm thick aluminum plate in an immersion tank (normal incidence, 7.5 MHz probe frequency, 100 MHz sampling frequency), a Wiener filtered signal, a single AR order extrapolation result, a ranged AR order extrapolation result, and an analytic signal magnitude of the single AR order extrapolation result, respectively. This example is demonstrative of the enhancement method described herein because the original echoes are overlapping, such that no estimate of plate thickness from the original signal would otherwise be possible.

TABLE 2

| | | Mean | | Mean | |
|---|---|---|---|---|---|
| | Actual | Thickness | | Thickness | |
| | Thick- | From Single | Differ- | From Ranged | Differ- |
| Material | ness | AR Order | ence | AR Order | ence |
| Aluminum | 3.14 | 3.1812 | +0.04 | 3.1644 | +0.02 |
| Aluminum | 1.59 | 1.5606 | −0.03 | 1.5606 | −0.03 |
| Aluminum | 0.82 | 0.8103 | −0.01 | 0.8103 | −0.01 |
| Galvanized | 1.48 | 1.4628 | −0.02 | 1.4768 | 0.00 |
| Steel | | | | | |
| Stainless | 0.42 | Unreadable | — | Unreadable | — |
| Steel | | | | | |

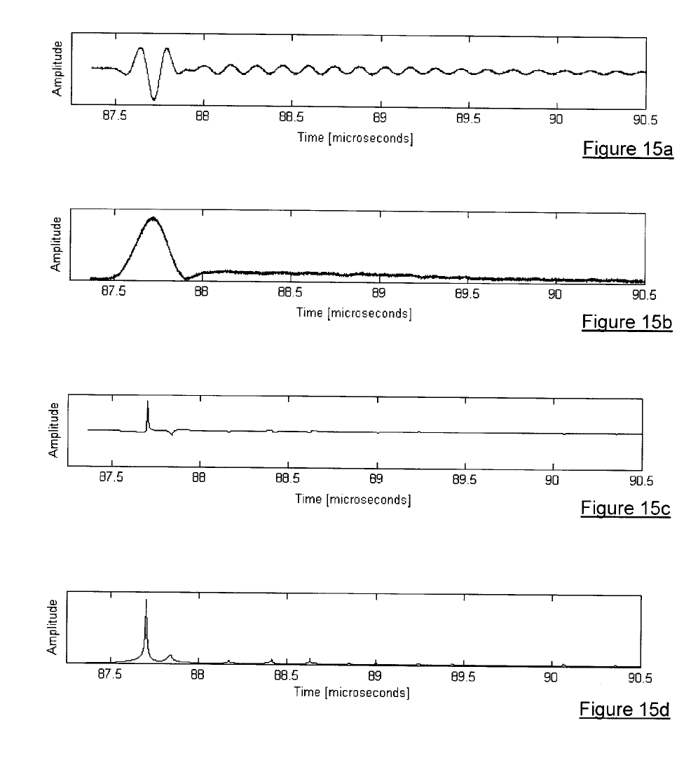

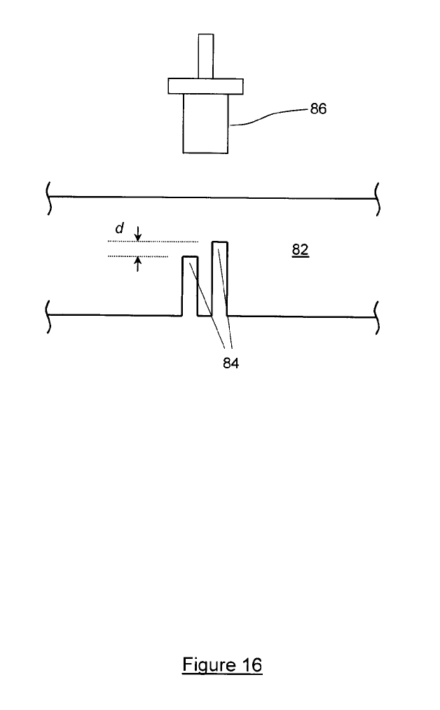

The signal from the stainless steel plate could not be improved to enable a measurement of the specimen thickness at a sampling frequency of 100 MHz. However, with the sampling frequency raised to 250 MHz, it was possible to resolve the backwall echoes to within about a quarter of a millimeter, as shown in Table 3 below. Table 3 shows results of the flat plate immersion test with a 7.5 MHz probe and a sampling frequency of 250 MHz at normal incidence (all distances are in mm; a negative distance implies the ultrasonic signal undersized the actual distance). As can be seen, the accuracy is poorer than that achieved for the other plates. However, no thickness measurement at all was possible from the original signal, and the signal processing improved the signal quality enough to enable a measurement to be taken on either the standard signal or analytic signal magnitude, as shown in FIG. 14. FIGS. 15a to 15d are graphical plots of a measured ultrasonic scan of a 0.42 mm thick stainless steel plate in an immersion tank (normal incidence, 7.5 MHz probe frequency, 100 MHz sampling frequency), an analytic signal magnitude of the measured signal, a ranged AR order extrapolation result, and an analytic signal magnitude of the ranged AR order extrapolation result, respectively. Note that no measurement of the plate thickness could be taken from the signal in FIG. 15a.

TABLE 3

| | | Mean | | Mean | |
|---|---|---|---|---|---|
| | Actual | Thickness | | Thickness | |
| | Thick- | From Single | Differ- | From Ranged | Differ- |
| Material | ness | AR Order | ence | AR Order | ence |
| Aluminum | 3.14 | 3.1400 | 0.00 | 3.1400 | 0.00 |
| Aluminum | 1.59 | 1.5953 | +0.01 | 1.6206 | +0.03 |
| Galvanized | 1.48 | 1.4282 | −0.05 | 1.4387 | −0.04 |
| Steel | | | | | |
| Stainless | 0.42 | 0.6471 | +0.23 | 0.6775 | +0.26 |
| Steel | | | | | |

The difficulty in obtaining an accurate measurement of plate thickness has often been observed when the amplitude of the frontwall echo is much larger than that of the first backwall echo. The very strong frontwall echo is well defined, but the rest of the signal is filtered out as noise. Increasing the sampling frequency has been observed to improve the ability to resolve the backwall echoes in some instances.

The results obtained for the 5 MHz probe on the aluminum plates are shown in Tables 4 and 5 below. Table 4 shows results of flat plate immersion test with a 5 MHz probe and a sampling frequency of 100 MHz at normal incidence, and Table 5 shows results of a flat plate immersion test with a 5 MHz probe and a sampling frequency of 250 MHz at normal incidence (all distances are in mm, and a negative distance implies the ultrasonic signal undersized the actual distance). This lower frequency (and attendant longer wavelength) leads to greater overlap of backwall echoes compared to the 7.5 MHz waves featured in Tables 2 and 3. As a result, the extrapolation technique was not able to clearly resolve the backwall echoes for all the plates, such that only limited results are shown in Tables 4 and Table 5.

TABLE 4

| | | Mean | | Mean | |
|---|---|---|---|---|---|
| | Actual | Thickness | | Thickness | |
| | Thick- | From Single | Differ- | From Ranged | Differ- |
| Material | ness | AR Order | ence | AR Order | ence |
| Aluminum | 3.14 | 3.1362 | 0.00 | 3.1350 | −0.01 |
| Aluminum | 1.59 | 1.7160 | +0.13 | 1.7369 | +0.15 |
| Galvanized | 1.48 | 1.4230 | −0.06 | 1.4369 | −0.04 |
| Steel | | | | | |

TABLE 5

| | | Mean | | Mean | |
|---|---|---|---|---|---|
| | Actual | Thickness | | Thickness | |
| | Thick- | From Single | Differ- | From Ranged | Differ- |
| Material | ness | AR Order | ence | AR Order | ence |
| Aluminum | 1.59 | 1.6038 | 0.01 | 1.5981 | 0.01 |
| Stainless | 0.42 | 0.3943 | 0.03 | 0.3943 | 0.03 |
| Steel | | | | | |

The flat plate immersion experiments with the 5 MHz transducer still show the algorithm's ability to characterize multiple internal echoes to within hundredths of a millimeter, in most cases. A comparison between results using a single value of p and a range of values for p when performing the signal enhancement showed no discernable difference.

### Example 4

In this example, a test specimen 82 was created for testing the signal processing scheme on single A-Scan images. Specimen 82 featured six pairs 84 of flat bottom holes. The holes of each pair 84 were drilled parallel to each other at a centre-to-centre distance of 1.5 mm. For each of the six pairs of holes, the relative difference in depth, d, was very small, as shown in FIG. 16. The depths of these holes were offset successively closer in each pair at distances listed in Table 6 below. Table 6 shows the results of ultrasonic measurements of a double flat-bottomed hole specimen using a sampling frequency of 500 MHz (all distances are in mm; a negative distance implies the ultrasonic signal undersized the actual distance).

TABLE 6

| Difference | Single AR | | Ranged AR | |
|---|---|---|---|---|
| In Hole | Order | | Order | |
| Depth, d | Distance | Difference | Distance | Difference |
| 0.9144 | 0.9068 | −0.0076 | 0.9103 | −0.0041 |
| 0.7874 | 0.8109 | +0.0235 | 0.8109 | +0.0235 |
| 0.6223 | 0.6295 | +0.0072 | 0.6295 | +0.0072 |
| 0.4953 | 0.5811 | +0.0858 | 0.5811 | +0.0858 |
| 0.4191 | 0.3749 | −0.0442 | 0.3749 | −0.0442 |
| 0.2794 | 0.3099 | +0.0305 | 0.3099 | +0.0305 |

When an ultrasonic probe 86 was used to examine the flat bottoms of each pair 84 of holes from the opposite surface, the return signal featured two very weak closely-spaced echoes. There was increasing overlap observed between these two echoes for the pairs of holes that are closest in depth. The goal of this experiment was to resolve these two echoes, and to observe and understand the limitations of the signal processing under these demanding conditions. The results of the tests are summarized in Table 6. The reference echo was taken from a clean backwall of the specimen 82, and away from the pair 84 of drilled holes.

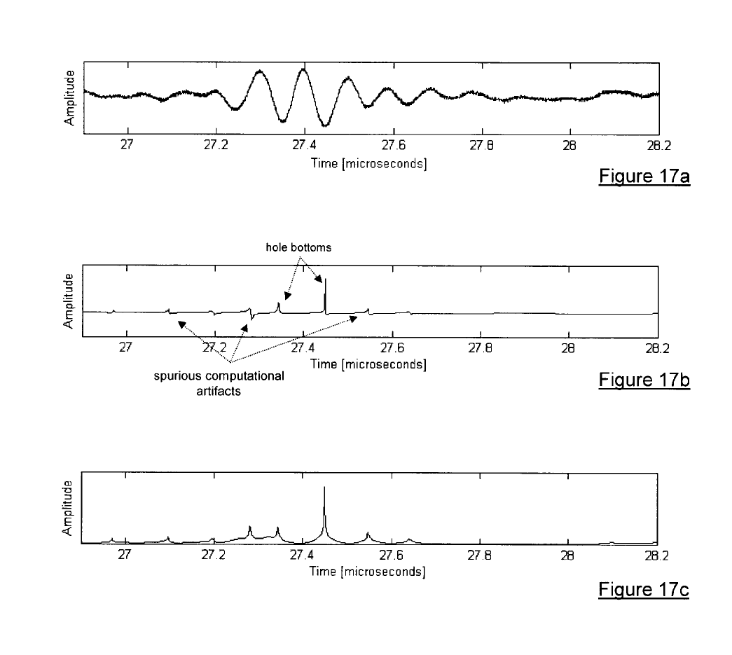

The signal processing methodology demonstrates the ability to aid the measurement of the distance between the hole faces to within less than a tenth of a millimeter. However, for many signals it becomes very difficult to distinguish the hole echoes from spurious spikes or computational artifacts that may appear in the processed signal. This difficulty is caused by the fact that the echoes of interest have a very low signal-to-noise ratio, and thus a high sampling frequency is needed to obtain a suitable signal for processing. As the sampling frequency is increased, the width of the frequency window being used for the extrapolation operation is decreased, and this is desirable for processing weak signals. In some of the cases, identifying the echoes from the hole bottoms was dependant on a manual examination of the final non-rectified signal where the polarity of the echoes provided clues to their origin, as shown in FIGS. 17a to 17c. FIGS. 17a to 17c are graphical plots of a measured ultrasonic scan of a specimen having two flat bottomed holes with a depth difference d=0.2794 mm (immersion tank, 7.5 MHz probe, sample frequency 500 MHz), a ranged AR order extrapolation result, and an analytic signal magnitude of the ranged AR order extrapolation result, respectively.

According to the signal processing methodology, a value of the AR order number, p, for “noisy” signals will be selected that is comparable to the number of points in the frequency window for sampling frequencies used in this experiment, as shown in Table 7 below. Table 7 shows a comparison of the AR order numbers and the number of points in the frequency window (“FW”) for the enhancement of the double flat bottomed hole experiment. In addition to extrapolating most of the information contained within the frequency window, the signal processing methodology will therefore also extrapolate most of the noise.

TABLE 7

| | Difference | Number of | | |
|---|---|---|---|---|
| Pair | In Hole | Data Points | | |
| Number | Depth | In FW | pₘᵢₙ | pₘₐₓ |
| 1 | 0.9144 | 17 | 16 | 17 |
| 2 | 0.7874 | 21 | 15 | 20 |
| 3 | 0.6223 | 12 | 11 | 12 |
| 4 | 0.4953 | 11 | 10 | 11 |
| 5 | 0.4191 | 12 | 11 | 12 |
| 6 | 0.2794 | 24 | 15 | 20 |

No significant difference was found between extrapolations using a range of p and those using a single value of p. However, this may be due in part the fact that some ranges contained as few as two values for p, as shown in Table 7. It should also be noted that there was no observable correlation between the difference in hole depth, d, and the number of data points in the FW. It is suggested that the amount of noise determines the value of m and n to a greater degree than the amount of overlap between echoes.

### Example 5

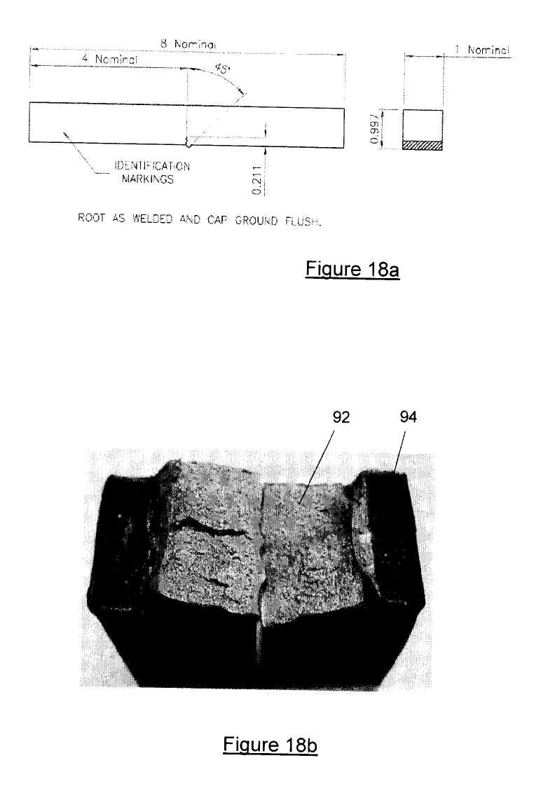

In this example, an Olympus NDT Omniscan™ was used, which is a portable phased array device that is marketed mainly for ultrasonic inspection of welds. To study the applicability of the algorithm to girth weld inspection, special welded specimens were fabricated to test the signal processing methodology with S-Scan imaging. Six bars, each with a cross sectional weld were created with a manufactured defect, namely a through-wall crack running along the surface of the weld, as shown in FIG. 18a. The depth of the crack was varied between specimens, and with values of 5%, 10%, 15%, 20%, 25%, and 30% of the specimen thickness. After the experiments, the specimens were broken along the weld to get an accurate destructive measure of the defects' depths. The specimens were first heated at 350° F. for two hours and allowed to cool naturally in order to oxidize the crack faces, as shown in FIG. 18b.

The OmniScan™ was used to collect ultrasonic echo data from the cracks. The measurements were taken using two models of phased array probe. The first, a 10L64-11 probe (10 MHZ, 64 elements, linear configuration), the second, a 5L16-A1 probe (5 MHz, 16 elements, linear configuration). Of these two probes, it was believed that the 10L64-11 probe was better suited to the inspection of these specimens, and thus, was expected to yield more accurate information from the ultrasonic echo signals. For each experiment, a 45° shear wedge was used, with a phased array angle sweep of between 45° and 70° relative to the specimen surface.

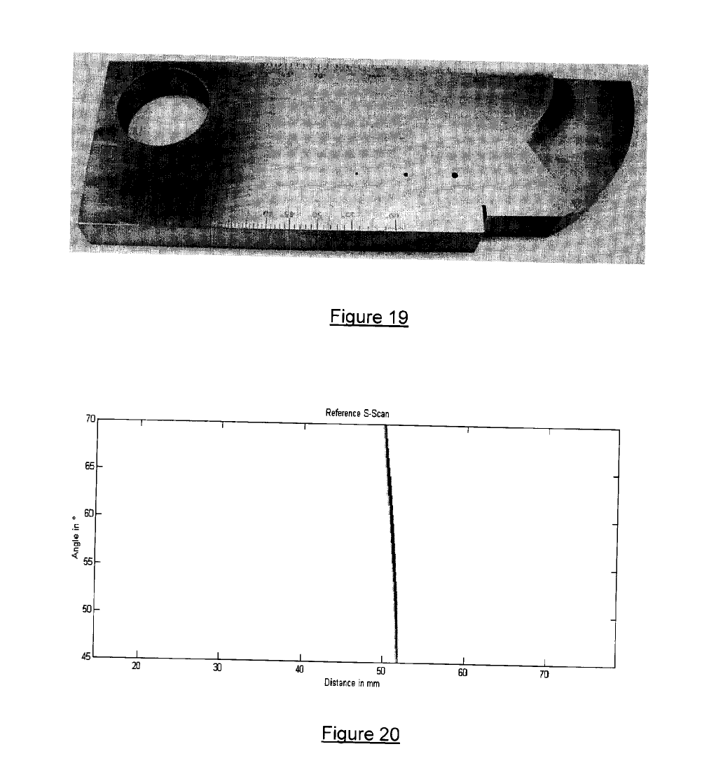

The goal of this experiment was to determine if the signal processing methodology could process an S-Scan image collected with the OmniScan instrument, and produce more accurate estimates of crack depth than could be derived from direct analysis of the raw signal. A set of reference echoes was obtained by utilizing the curved section of a calibration block, shown in FIG. 19, to produce clean backwall echoes at the same angles used to produce each S-Scan, as plotted in FIG. 20.

An S-Scan can be displayed in several ways. Although the “true-depth” display is most common, it proved difficult to render in the programming software (MatLab®) used. Therefore, a “sound-path” configuration has been used for all S-Scan figures herein. The depth of any two points on a sound-path rendering can be converted to true depth through the formula:

depth=s₂ cos(θ₂)−s₁ cos(θ) (14)

where s is the sound path distance, and θ is the beam angle of incidence.

The results for the experiments are shown in Tables 8 and 9 below. Table 8 shows results of measurements taken with a 10L64-11 probe with a 45° shear wave wedge, and Table 9 shows results of measurements taken with a 5L16-A1 probe with a 45° shear wave wedge (all distances are in mm).

TABLE 8

| Specimen ID: | Nominal crack | Crack Depth | Crack Depth | Crack Depth | |
|---|---|---|---|---|---|
| Approximate | depth on | Estimated | from Processed | from Processed | Destructive |
| fractional | specimen | from Raw S- | Signal: Single | Signal: Ranged | Measurement |
| Crack Depth | drawing | Scan | AR Order | AR Order | of Crack Depth |
| 30% | 7.6200 | 6.9567 | 7.5988 | 7.5546 | 7.66 |
| 25% | 6.5532 | 6.9867 | 6.3443 | 6.3279 | 6.56 |
| 20% | 5.3594 | 5.6699 | 5.3739 | 5.0778 | 5.32 |
| 15% | 3.7846 | 3.3899 | 3.4816 | 4.0602 | 3.80 |
| 10% | 2.3876 | 2.7256 | 2.3305 | 2.2850 | 2.36 |
| 5% | 1.3970 | 1.3725 | 1.4238 | 1.3795 | 1.40 |

TABLE 9

| Specimen ID: | | Crack Depth | Crack Depth | Crack Depth | |
|---|---|---|---|---|---|
| Approximate | Nominal crack | Estimated | from Processed | from Processed | Destructive |
| Fractional | depth on | from Raw S- | Signal: Single | Signal: Ranged | Measurement |
| Crack Depth | specimen | Scan | AR Order | AR Order | of Crack Depth |
| 30% | 7.6200 | 7.3366 | 7.7541 | 6.9914 | 7.66 |
| 25% | 6.5532 | 7.1485 | 6.7890 | 6.7709 | 6.56 |
| 20% | 5.3594 | 5.0039 | 5.0848 | 5.0876 | 5.32 |
| 15% | 3.7846 | 3.9986 | 3.6014 | 3.5946 | 3.80 |
| 10% | 2.3876 | 2.7256 | 2.4763 | 2.3632 | 2.36 |
| 5% | 1.3970 | 2.0261 | 2.1052 | 2.1103 | 1.40 |

On examining the error in crack depth estimates based on the single AR order and ranged AR order signal processing techniques, as shown in Tables 10 and 11 below, there is again no clear indication that using a range of p values is consistently better than using a single value of p. Table 10 shows differences between distances measured with ultrasonic techniques (10 MHz transducer) and actual distance measured after specimen destruction from Table 8, and Table 11 shows differences between distances measured with ultrasonic techniques (5 MHz transducer) and actual distance measured after specimen destruction from Table 9 (the technique with the smallest absolute difference is shown in bold; all distances are in mm, and a negative distance implies the ultrasonic signal undersized the actual distance). It may be seen that the processing of the scans taken using the 10L64-11 probe yielded more accurate results than the 5L16-A1 probe.

TABLE 10

| Specimen ID: | | Crack Depth Error | |
|---|---|---|---|
| Approximate | Crack Depth Error | from Processed | Crack Depth Error |
| fractional | Estimated from | Signal: Single | from Processed Signal: |
| Crack Depth | Raw S-Scan | AR Order | Ranged AR Order |
| 30% | −0.7033 | −0.0612 | −0.1054 |
| 25% | +0.4267 | −0.2157 | −0.2321 |
| 20% | +0.3499 | +0.0539 | −0.2422 |
| 15% | −0.4101 | −0.3184 | +0.2602 |
| 10% | +0.3656 | −0.0295 | −0.0750 |
| 5% | −0.0275 | +0.0238 | −0.0205 |

The enhancements of the S-Scan data taken with the 10L64-11 probe (see Table 10) yielded estimates of crack depth that are generally accurate to within 0.3 millimeters, compared with the raw S-Scan signal which is generally accurate only to within 0.7 millimeters. There was no discernable difference between the results obtained using a ranged value of p and those obtained using a single value of p.

TABLE 11

| Specimen ID: | | Crack Depth Error | |
|---|---|---|---|
| Approximate | Crack Depth Error | from Processed | Crack Depth Error |
| fractional | Estimated from | Signal: Single | from Processed Signal: |
| Crack Depth | Raw S- Scan | AR Order | Ranged AR Order |
| 30% | −0.3234 | +0.0941 | −0.6686 |
| 25% | +0.5885 | +0.2290 | +0.2109 |
| 20% | −0.3161 | −0.2352 | −0.2324 |
| 15% | +0.1986 | −0.1986 | −0.2054 |
| 10% | +0.2842 | +0.1163 | −0.0032 |
| 5% | +0.6261 | +0.7052 | +0.7103 |

Autoregressive extrapolation of the data obtained using the 5L16-A1 probe did not always yield more accurate estimates of crack depth than the raw signal, see for example the 5% specimen in Table 11. However such deterioration in sizing ability occurred only on this one specimen with the smallest crack. The data suggests that the 10L64-11 probe is in fact superior to that of the 5L16-A1 probe for this application in terms of echo resolution, and also shows that enhancements made from the former probe yielded more accurate results than the final enhancements made from the 5L16-A1 probe.

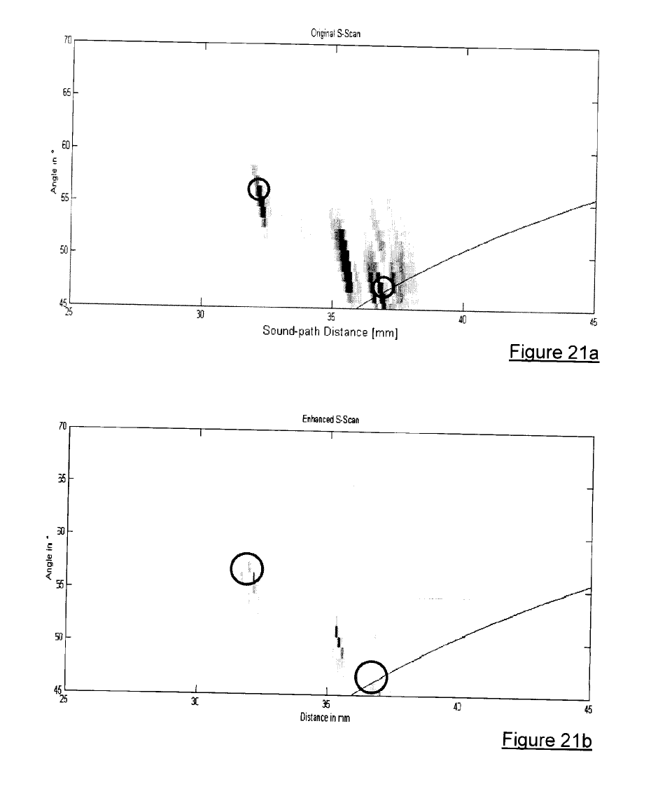

A typical S-Scan and its enhancement are shown in FIGS. 21a and 21b, respectively. FIG. 21a shows the captured rectified S-Scan with the 10L64-11 probe of the 30% cracked bar specimen (raw signal, as currently displayed in field use of the OmniScan™ instrument). Circles have been superimposed to show the crack tip and the echo originating from the point where the crack opens to the specimen surface, a positional estimate of which is indicated by the black line. The echoes seen between the crack tip and corner trap are other faces of the crack between these two points that reflect or diffract the ultrasonic beam. The depth distance between the two points is calculated using Equation 14.

Although embodiments have been described above with reference to the accompanying drawings, those of skill in the art will appreciate that variations and modifications may be made without departing from the spirit and scope thereof as defined by the appended claims.

1. An ultrasonic signal processing method comprising:
deconvolving the received ultrasonic signal to yield a filtered signal;
determining autoregressive extrapolation parameters based on frequency amplitude fluctuations of the filtered signal within a frequency range over which a corresponding reference signal has a high signal-to-noise ratio; and
carrying out an autoregressive spectral extrapolation of the filtered signal using the autoregressive extrapolation parameters to yield an enhanced ultrasonic signal.

2. The method of claim 1, wherein the ultrasonic signal is deconvolved by Wiener filtering.

3. The method of claim 1, wherein the determining comprises:
defining an initial estimate of the frequency range as a range of frequency over which the corresponding reference signal has a high signal-to-noise ratio;
fitting a polynomial approximation to the filtered signal within the initial estimate of the frequency range;
defining ranges of possible values of frequency window boundaries m and n from the polynomial approximation according to a first set of criteria;
determining a quantitative measure of frequency amplitude fluctuations of the filtered signal relative to the polynomial approximation within a maximum frequency range defined by boundaries m and n;
defining initial values of an autoregressive (AR) order p according to the quantitative measure;
determining initial values of boundaries m and n based on a quantitative measure of frequency amplitude fluctuations of the filtered signal relative to the polynomial approximation within frequency ranges defined by a maximum initial value of AR order p and the ranges of possible values of frequency window boundaries m and n;
defining final values of AR order p according to the initial values of boundaries m and n and to a second set of criteria; and
determining final values of boundaries m and n based on a quantitative measure of frequency amplitude fluctuations of the filtered signal relative to the polynomial approximation within frequency ranges defined by a maximum final value of AR order p and the ranges of possible values of frequency window boundaries m and n, the final values of boundaries m and n and AR order p being the autoregressive extrapolation parameters.

4. The method of claim 3, wherein the carrying out comprises using a single final value of AR order p.

5. The method of claim 3, wherein the carrying out comprises using a plurality of final values of AR order p, carrying out an autoregressive spectral extrapolation using at least two final values of AR order p in the plurality to yield a plurality of autoregressive extrapolations, and averaging the plurality of autoregressive spectral extrapolations to yield a single autoregressive spectral extrapolation.

6. An ultrasonic scanning system comprising:
a processor;
a transmitting probe in communication with the processor, the transmitting probe being configured for emitting an ultrasonic signal into a specimen in accordance with instructions from the processor; and
a receiving probe in communication with the processor, the receiving probe being configured for receiving a return ultrasonic signal and for communicating the received ultrasonic signal to the processor, wherein the processor is configured to:
transform the received ultrasonic signal into a frequency domain;
deconvolve the received ultrasonic signal by Wiener filtering to yield a filtered signal;
determine autoregressive extrapolation parameters based on frequency amplitude fluctuations of the filtered signal within a frequency range over which a corresponding reference signal has a high signal-to-noise ratio; and
carry out an autoregressive spectral extrapolation of the filtered signal using the autoregressive extrapolation parameters to yield an enhanced ultrasonic signal.

7. An ultrasonic measurement system comprising:
a processor; and
an ultrasonic probe in communication with the processor, the ultrasonic probe being configured for generating a transmitted ultrasonic signal in accordance with instructions from the processor, for receiving a return ultrasonic signal and for communicating the received ultrasonic signal to the processor;
the processor being configured for enhancing the received ultrasonic signal by:
transforming the received ultrasonic signal into a frequency domain;
deconvolving the received ultrasonic signal by Wiener filtering to yield a filtered signal;
determining autoregressive extrapolation parameters based on frequency amplitude fluctuations of the filtered signal within a frequency range over which a corresponding reference signal has a high signal-to-noise ratio; and
carrying out an autoregressive spectral extrapolation of the filtered signal using the autoregressive extrapolation parameters to yield an enhanced ultrasonic signal.

8. An ultrasonic measurement system comprising:
a processor;
an ultrasonic pulser in communication with the processor;
a transmitting probe in communication with the pulser for generating a transmitted ultrasonic signal in response to the pulser;
a receiving probe for receiving a return ultrasonic signal;
a received signal preprocessor in communication with the processor and the receiving probe, the preprocessor comprising an analog-digital converter for digitizing the received ultrasonic signal communicated by the receiving probe, the preprocessor communicating the digitized received ultrasonic signal to the processor, the processor enhancing the digitized received ultrasonic signal by:
transforming the digitized received ultrasonic signal into a frequency domain;
deconvolving the digitized received ultrasonic signal by Wiener filtering to yield a filtered signal;
determining autoregressive extrapolation parameters based on frequency amplitude fluctuations of the filtered signal within a frequency range over which a corresponding reference signal has a high signal-to-noise ratio; and
carrying out an autoregressive spectral extrapolation of the filtered signal using the autoregressive extrapolation parameters to yield an enhanced ultrasonic signal.

9. An ultrasonic measurement system comprising:
a processor;
an ultrasonic pulser in communication with the processor;
an ultrasonic probe in communication with the pulser, the ultrasonic probe being configured for transmitting an ultrasonic signal and for receiving a transmitted ultrasonic signal;
a received signal preprocessor in communication with the processor and the ultrasonic probe, the preprocessor comprising an analog-digital converter for digitizing a received ultrasonic signal communicated by the receiving probe, the preprocessor communicating a digitized received ultrasonic signal to the processor, the processor enhancing the digitized received ultrasonic signal by:
transforming the digitized received ultrasonic signal into a frequency domain;
deconvolving the digitized received ultrasonic signal by Wiener filtering to yield a filtered signal;
determining autoregressive extrapolation parameters based on frequency amplitude fluctuations of the filtered signal within a frequency range over which a corresponding reference signal has a high signal-to-noise ratio; and
carrying out an autoregressive spectral extrapolation of the filtered signal using the autoregressive extrapolation parameters to yield an enhanced ultrasonic signal.

10. The system of claim 6, wherein the processor during the determining is configured to:
define an initial estimate of the frequency range as a range of frequency over which the corresponding reference signal has a high signal-to-noise ratio;
fit a polynomial approximation to the filtered signal within the initial estimate of the frequency range;
define ranges of possible values of frequency window boundaries m and n from the polynomial approximation according to a first set of criteria;
determine a quantitative measure of frequency amplitude fluctuations of the filtered signal relative to the polynomial approximation within a maximum frequency range defined by boundaries m and n;
define initial values of an autoregressive (AR) order p according to the quantitative measure;
determine initial values of boundaries m and n based on a quantitative measure of frequency amplitude fluctuations of the filtered signal relative to the polynomial approximation within frequency ranges defined by a maximum initial value of AR order p and the ranges of possible values of frequency window boundaries m and n;
define final values of AR order p according to the initial values of boundaries m and n and to a second set of criteria; and
determine final values of boundaries m and n based on a quantitative measure of frequency amplitude fluctuations of the filtered signal relative to the polynomial approximation within frequency ranges defined by a maximum final value of AR order p and the ranges of possible values of frequency window boundaries m and n, the final values of boundaries m and n and AR order p being the autoregressive extrapolation parameters.

11. The system of claim 10, wherein the processor is configured to carry out the autoregressive spectral extrapolation using a single final value of AR order p.

12. The system of claim 10, wherein the processor is configured to carry out the autoregressive spectral extrapolation using at least two final values of AR order p in a plurality of final values of AR order p to yield a plurality of autoregressive extrapolations, and averaging the plurality of autoregressive spectral extrapolations to yield a single autoregressive spectral extrapolation.

13. A method of enhancing an ultrasonic signal, the method comprising:
receiving a first signal from a specimen;
receiving a second signal from a specimen, the second signal being a reference signal, the first and second signals being frequency domain ultrasonic signals;
deconvolving the first signal to yield a filtered first signal;
determining autoregressive extrapolation parameters based on frequency amplitude fluctuations of the filtered first signal within a frequency range over which the second signal has a high signal-to-noise ratio; and
carrying out an autoregressive spectral extrapolation of the filtered first signal using the autoregressive extrapolation parameters to yield an enhanced first signal.

14. The method of claim 13, wherein the deconvolving comprises deconvolving by Wiener filtering.

15. The method of claim 13, wherein the determining comprises:
defining an initial estimate of the frequency range as a range of frequency over which the corresponding reference signal has a high signal-to-noise ratio;
fitting a polynomial approximation to the filtered signal within the initial estimate of the frequency range;
defining ranges of possible values of frequency window boundaries m and n from the polynomial approximation according to a first set of criteria;
determining a quantitative measure of frequency amplitude fluctuations of the filtered signal relative to the polynomial approximation within a maximum frequency range defined by boundaries m and n;
defining initial values of an autoregressive (AR) order p according to the quantitative measure;
determining initial values of boundaries m and n based on a quantitative measure of frequency amplitude fluctuations of the filtered signal relative to the polynomial approximation within frequency ranges defined by a maximum initial value of AR order p and the ranges of possible values of frequency window boundaries m and n;
defining final values of AR order p according to the initial values of boundaries m and n and to a second set of criteria; and
determining final values of boundaries m and n based on a quantitative measure of frequency amplitude fluctuations of the filtered signal relative to the polynomial approximation within frequency ranges defined by a maximum final value of AR order p and the ranges of possible values of frequency window boundaries m and n, the final values of boundaries m and n and AR orderp being the autoregressive extrapolation parameters.

16. The method of claim 15, wherein the carrying out comprises using a single final value of AR order p.

17. The method of claim 15, wherein the carrying out comprises using a plurality of final values of AR orderp, carrying out an autoregressive spectral extrapolation using at least two the final values of AR order p in the plurality to yield a plurality of autoregressive spectral extrapolations, and averaging the plurality of autoregressive spectral extrapolations to yield a single autoregressive extrapolation.

18. A non-transitory processor readable memory having recorded thereon statements and instructions for execution by a processor to carry out the method of claim 13.

19. The system of claim 7, wherein the processor during the determining is configured to:
define an initial estimate of the frequency range as a range of frequency over which the corresponding reference signal has a high signal-to-noise ratio;
fit a polynomial approximation to the filtered signal within the initial estimate of the frequency range;
define ranges of possible values of frequency window boundaries m and n from the polynomial approximation according to a first set of criteria;
determine a quantitative measure of frequency amplitude fluctuations of the filtered signal relative to the polynomial approximation within a maximum frequency range defined by boundaries m and n;
define initial values of an autoregressive (AR) order p according to the quantitative measure;
determine initial values of boundaries m and n based on a quantitative measure of frequency amplitude fluctuations of the filtered signal relative to the polynomial approximation within frequency ranges defined by a maximum initial value of AR order p and the ranges of possible values of frequency window boundaries m and n;
define final values of AR order p according to the initial values of boundaries m and n and to a second set of criteria; and
determine final values of boundaries m and n based on a quantitative measure of frequency amplitude fluctuations of the filtered signal relative to the polynomial approximation within frequency ranges defined by a maximum final value of AR order p and the ranges of possible values of frequency window boundaries m and n, the final values of boundaries m and n and AR order p being the autoregressive extrapolation parameters.

20. The system of claim 8, wherein the processor during the determining is configured to:
define an initial estimate of the frequency range as a range of frequency over which the corresponding reference signal has a high signal-to-noise ratio;
fit a polynomial approximation to the filtered signal within the initial estimate of the frequency range;
define ranges of possible values of frequency window boundaries m and n from the polynomial approximation according to a first set of criteria;
determine a quantitative measure of frequency amplitude fluctuations of the filtered signal relative to the polynomial approximation within a maximum frequency range defined by boundaries m and n;
define initial values of an autoregressive (AR) order p according to the quantitative measure;
determine initial values of boundaries m and n based on a quantitative measure of frequency amplitude fluctuations of the filtered signal relative to the polynomial approximation within frequency ranges defined by a maximum initial value of AR order p and the ranges of possible values of frequency window boundaries m and n;
define final values of AR order p according to the initial values of boundaries m and n and to a second set of criteria; and
determine final values of boundaries m and n based on a quantitative measure of frequency amplitude fluctuations of the filtered signal relative to the polynomial approximation within frequency ranges defined by a maximum final value of AR order p and the ranges of possible values of frequency window boundaries m and n, the final values of boundaries m and n and AR order p being the autoregressive extrapolation parameters.

21. The system of claim 9, wherein the processor during the determining is configured to:
define an initial estimate of the frequency range as a range of frequency over which the corresponding reference signal has a high signal-to-noise ratio;
fit a polynomial approximation to the filtered signal within the initial estimate of the frequency range;
define ranges of possible values of frequency window boundaries m and n from the polynomial approximation according to a first set of criteria;
determine a quantitative measure of frequency amplitude fluctuations of the filtered signal relative to the polynomial approximation within a maximum frequency range defined by boundaries m and n;
define initial values of an autoregressive (AR) order p according to the quantitative measure;
determine initial values of boundaries m and n based on a quantitative measure of frequency amplitude fluctuations of the filtered signal relative to the polynomial approximation within frequency ranges defined by a maximum initial value of AR order p and the ranges of possible values of frequency window boundaries m and n;
define final values of AR order p according to the initial values of boundaries m and n and to a second set of criteria; and
determine final values of boundaries m and n based on a quantitative measure of frequency amplitude fluctuations of the filtered signal relative to the polynomial approximation within frequency ranges defined by a maximum final value of AR order p and the ranges of possible values of frequency window boundaries m and n, the final values of boundaries m and n and AR order p being the autoregressive extrapolation parameters.
